(19) United States
(12) Patent Application Publication
(10) Pub. No.: US 2007/0055148 A1
(43) Pub. Date: Mar. 8, 2007

(54) Method for determining the dispersion behavior of a contrast agent bolus

(75) Inventor: Klaus Klingenbeck-Regn, Numberg (DE)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Aktiengesellschaft

(21) Appl. No.: 11/516,110

(22) Filed: Sep. 6, 2006

(30) Foreign Application Priority Data
Sep. 6, 2005 (DE) 10 2005 042 328.0

(51) Int. Cl. A61B 6/00

(52) U.S. Cl. 600/431

(57) ABSTRACT

A method for determining the dispersion behavior of a contrast agent in the blood vessel system of an object for a rotational angiography procedure, comprising:

introducing the contrast agent into the blood vessel system and determining the time t1 of said introduction,

producing preparatory images of at least a part of the vascular system in order to determine the time curve for the uptake of the contrast agent in the arterial vascular system and, determining a time t2 for recording a rotational angiography image of an arterial phase,

producing preparatory images of at least a part of the vascular system in order to determine the time curve for the uptake of the contrast agent in the venous vascular system and, determining a time t3 for recording a rotational angiography image of a venous phase, and

determining the differences in the times t1, t2 and t3 for the rotational angiography.

### CROSS REFERENCE TO RELATED APPLICATIONS

This application claims priority of German application No. 10 2005 042 328.0 filed Sep. 6, 2005, which is incorporated by reference herein in its entirety.

### FIELD OF THE INVENTION

The invention relates to a method for determining the dispersion behavior of a contrast agent bolus in the blood vessel of an object under investigation for a rotational angiography procedure that is to be performed.

### BACKGROUND OF THE INVENTION

Modern C-arm systems enable a three-dimensional representation of vascular trees to be obtained. Toward that end, the C-arm is rotated around the patient in order to record two-dimensional projections from which the anatomical structure of interest is subsequently reconstructed three-dimensionally. For this purpose mathematical algorithms are used by means of which it is possible, as part of what is referred to as cone-beam computer tomography, for example, to determine the three-dimensional reconstruction.

For the purpose of performing rotational angiography procedures it is necessary, in order to clarify certain medical questions, to determine the times at which the contrast agent reaches the arteries or veins respectively, which is to say is taken up in the respective vascular system. Information of this kind is important, for example, in order to establish the binding of an arterio-venous malformation to the arterial and the venous vascular system in the course of the angiography procedure. In the past, however, the times have been merely estimated, from empirical values for example. But if a contrast agent is injected into the vascular pathway of an object under investigation, images that are taken show a change in color in the arteries and veins due to the contrast agent according to the circulatory condition of the patient, for example according to heart function, at times that vary significantly from individual to individual.

If said times are estimated incorrectly, unsatisfactory results can be produced in the course of the rotational angiography procedure, with images on which the separation between the arterial and the venous system in particular is not clear. As a consequence the evaluation of the recorded three-dimensional images is rendered more difficult.

### SUMMARY OF THE INVENTION

The object underlying the invention is therefore to set forth a method for determining the dispersion behavior of a contrast agent bolus in the blood vessel system of an object under investigation for a rotational angiography procedure that is to be performed, which method allows the dispersion behavior to be determined in an optimal manner.

In order to achieve this object, a method of the aforementioned kind is provided which comprises the following steps:

Introduction of the contrast agent into the blood vessel system of the object under investigation and determination of the time t1 of said introduction,

Production of preparatory images of at least a part of the vascular system in order to determine the time curve for the uptake of the contrast agent bolus in the arterial vascular system and, as a function thereof, determination of a time t2 for recording a rotational angiography image of an arterial phase,

Production of preparatory images of at least a part of the vascular system in order to determine the time curve for the uptake of the contrast agent bolus in the venous vascular system and, as a function thereof, determination of a time t3 for recording a rotational angiography image of a venous phase, and

Determination of the differences in the identified times t1, t2 and t3 to be held available for the rotational angiography procedure.

Thus, a contrast agent is first injected into the blood vessel system of a patient and in this way enters the vascular pathway. The start of or, as the case may be, a characteristic time during the contrast agent injection is recorded by storing the associated time t1 of the introduction.

Next, preparatory images are taken which show at least a part of the vascular system for example in a specified region of interest to be investigated or in the area of an organ of the human or animal object under investigation in order to determine, with the aid of said images, the time curve for the uptake of the contrast agent bolus in the arterial vascular system at least for a specific region. From this image sequence, which is produced e.g. as an angiogram, a time t2 is determined which is optimal for recording a rotational angiography image of the arterial phase. This takes place through observation of the staining of the arteries in the recorded image sequence. The image sequence can by all means cover a longer period of time, i.e. also the excretion of the contrast agent etc., so “uptake” should be understood here as a very expansive term. As a rule, however, the optimal time t2 is in the area of a maximum coloration. The specific time t2 is likewise stored.

Analogously thereto, preparatory images are taken in order to determine the time curve for the entry of the contrast agent bolus into the venous vascular system and where applicable the distribution in the system, from which images a time t3 is determined as the optimal time for recording rotational angiography images of a venous phase.

Differences are formed in each case from the determined times t1, t2 and t3, the differences t2−t1, t3−t1 and t3−t2 being stored for the subsequent three-dimensional recording of the arterial or, as the case may be, venous phase during the rotational angiography procedure. In this case the differences form delays that are to be set for the recording of rotational angiography images in a C-arm system. The differences or delays here are to be input as specifications of times that elapse for example between the injection of the contrast agent and its uptake in the arterial system, together with their amounts.

As a result of the taking of the test images before the actual rotational angiography procedure according to the inventive method, the times at which the arteries and the veins change color after an injection of contrast agent are optimally and individually determined for the particular patient and for the injected contrast agent etc. This individualization advantageously leads to three-dimensional images with optimal image results being obtained in the subsequent rotational angiography, it being possible in particular to achieve an optimal separation of the arterial and the venous system.

In this case the preparatory images can possibly be recorded as accelerated measurements in which a limited amount of image data is acquired. The only critical factor is that the optimal time for the subsequent three-dimensional image can be derived from the coloring of the vascular system. Moreover, the test images do not necessarily have to be produced using the same investigation modality or the same imaging methods as the subsequent images. This means that two-dimensional images can be adequate as preparatory images provided they show the vascular system with sufficient precision. It is, however, equally possible to perform a three-dimensional reconstruction even for the test images.

According to the invention the time curve for the uptake can be determined by tracking the coloration induced by the contrast agent. In this case use is made of the fact that the usual consequence of introducing contrast agent is that the vessels in images change in color in a specific manner when the bolus reaches the vessel and after a certain time return to the original coloration once the contrast agent bolus has been excreted. The injected contrast agents modify e.g. the signal that is recorded in the course of an x-ray examination or magnetic resonance examination in that they absorb the radiation more strongly or have paramagnetic properties, etc. An iodine-containing solution, for example, can be injected as the contrast agent, thereby rendering the vessels visible.

The preparatory images can be produced at least in part using subtraction angiography, more particularly digital subtraction angiography, and/or as native images.

Performing subtraction angiography, which is advantageously performed as digital subtraction angiography, it also being possible, of course, to perform analog subtraction angiography, is recommended for the situation in which static regions or, as the case may be, objects in the or of the object under investigation are to be the subject of the images. Cranial images should be mentioned here by way of example. The recorded images can be stored in digital form and subsequently the image objects which are free of contrast agent can be subtracted from the image objects that show contrast agent, thereby producing, as a result of this subtraction, a representation that can be readily evaluated.

In the case of non-static objects as part of the recorded images, in the area of the heart for example, native images can be taken as preparatory images. Said native images can be evaluated directly, without need for processing by subtraction or the like. Although the native images may be more difficult to evaluate, they avoid errors due to movements of the recorded objects or, as the case may be, areas of the body.

At least one time t2, t3 for preparatory images of a phase can be determined automatically, in particular through use of a digital subtraction angiography function for locating images of maximum contrast density. In order to perform the digital subtraction angiography, specific program means are provided in the control devices for, for example, suitably embodied C-arm systems, said program means having different functions. Through the use of a function which permits images with a maximum contrast density to be located automatically, the time associated with these images can be determined as a time that is optimal for the recording of the arterial or venous phase, without further action on the part of an operator.

Needless to say, a different method for automatically determining the times t2, t3 is also possible, for example by recourse to database information or certain image processing methods.

According to the invention the differences in the determined times t1, t2 and t3 are stored in a control device for the rotational angiography. There they are available directly for setting for the three-dimensional imaging to be performed subsequently. In addition it is of course possible to store the differences of the determined times or the times themselves directly in a central memory device for example for a plurality of examination equipment of a clinic or the like or to use an external storage medium such as a CD-Rom or the like. Storing the data in the control device for a rotational angiography procedure has the advantage that the data can be stored in such a way that a corresponding program means for performing the examination can locate the time data without further processing and initiate the corresponding setting of the C-arm system provided for recording the image.

The contrast agent can be introduced arterially or intravenously. In this case the manner of injection of the contrast agent will depend on the questions to be resolved by the examination as well as on the possibilities for injecting the agent into the patient. Depending on the type of injection into the arterial or venous system, it may be necessary to adapt the order in which the preparatory images are taken for determining the arterial or venous phase.

The contrast agent can also be introduced by manual and/or automatic injection. In this case one or more suitable injectors should be chosen in order to introduce the contrast agent for example partially automatically and in addition to inject it manually. As a rule either a manual or an automatic injection will be performed, depending on the amount to be injected and a specified injection rate.

The start of an injection can be determined as the time t1 of the introduction of the contrast agent. If an automatic injector is used, the time t1 can in this case be specified or defined by means of the setting, performed for example by a physician or technician, of said injector. Furthermore, said time t1 can often be read off subsequently on an automatic injector if the injector is embodied for storing specific injection information such as the time of day or suchlike. Depending on the type of the introduced contrast agent or, as the case may be, of the injection, further times apart from the start of the injection can be determined as the time ti for introducing the agent, for example the time at which half of the contrast agent has entered the body of the patient. In the case where a C-arm system used has an interface to an injector, it is possible that the start of the injection is integrated into an acquisition protocol for the production of images which is available in a control device.

In addition, further preparatory images for determining the time curve for the uptake of the contrast agent bolus in the vascular system can be generated and, as a function thereof, at least one further time for recording a rotational angiography image of at least one further phase, in particular a phase of the liver, can be determined and incorporated into the determination of the differences. Thus, a three-phase recording of the liver is recommended for particular medical questions in which an early arterial phase, a portal-venous phase and a late arterial phase are distinguished. Multiphase examinations of this type enable reliable location of lesions with the most varied blood supply types. In such a case a further time, for example a time t4 for an arterial late phase, must be determined in the course of the preparatory images. It is, of course, conceivable to determine further times in addition and include them in the forming of the differences. By determining the differences and programming these delays into a control device for the rotational angiography it is possible for the subsequent images to be taken at their respective optimal times or, as the case may be, in periods specified thereby.

In order to perform the subsequent rotational angiography procedure, the differences held in reserve can be set at least partially manually in a control device. An operator of the C-arm system, for example a physician, physicist or technician, can therefore set, for example, the difference t2−t1 by way of a corresponding input device so that after the time difference has elapsed the corresponding data acquisition is performed by rotation of the C-arm. Moreover the setting can be performed in such a way that the operator starts the recording of the image in accordance with the time difference, i.e. maintains the delays manually.

Furthermore, in order to perform the subsequent rotational angiography, the stored differences can be set at least partially automatically by a control device, in particular in the course of an executing protocol. For this purpose the C-arm system may have an automatic acquisition protocol in which the injection, the arterial phase and the venous phase are stored for the image sequence. The operator inputs the previously determined times or delays via a user interface or, alternatively, these are retrieved from the memory of the control device and set automatically, whereupon the C-arm system is controlled via the control device in such a way that the recording protocol executes automatically. Semiautomatic execution is also possible, wherein for example the delays for two phases are recorded and set automatically, while an operator inputs a further time for a third phase as necessary.

As already explained, in order to perform the subsequent rotational angiography the determined times t1, t2 and t3 and/or the stored differences can be input via a user interface of a control device. The user interface enables the operator to interact with a program means which controls the data acquisition in order to make changes if necessary, such as for example the input of the individual times determined here for recording the data of the individual phases, so that in contrast to a possibly present standard protocol an optimal image quality of the recorded images can be achieved.

According to the invention, in order to perform the subsequent rotational angiography the introduction of the contrast agent can be started automatically on the part of a control device, in particular with the aid of an interface provided for this purpose between the control device and an injector. With the aid of a program means of the control device a time for the injection of the contrast agent can be specified via the interface, for which purpose a corresponding signal is transmitted to an electronic injector, said signal possibly being received directly by the latter at the time of the desired start or being stored in a memory unit of the same in order to start the injection at a later time. An automatic injection is possible both for the production of the test images and for the subsequent rotational angiography, which may be able to execute fully automatically depending on the stored delays after an operator has specified the type of images to be taken or confirmed the image to be recorded for example by means of a click of the mouse or suchlike. The task of the operator thereafter consists essentially in monitoring the data acquisition.

The invention also relates to a rotational angiography device having a C-arm system for performing a rotational angiography procedure while determining the dispersion behavior of a contrast agent bolus as claimed in one of the preceding claims. The rotational angiography device according to the invention thus has means for determining the above described test images for determination of the times for recording the arterial, venous and, if necessary, further phases. Said determined differences or delays can subsequently be set on a control device of the rotational angiography device in order thereby to perform the rotational angiography at optimal times for ensuring a good image quality. It is therefore possible for example when an arterio-venous malformation is present to obtain image results which show an optimal separation of the arterial and the venous system.

A control device of the rotational angiography device can be embodied for at least partially automatic determination of the times t1, t2 and t3 and/or of the differences. The times can be derived automatically from the preparatory images with the aid of a program means which allows a corresponding image evaluation. They are subsequently stored in the control device which forms the differences t2−t1, t3−t1 and t3−t2 which are set for the subsequent rotational angiography possibly automatically, in certain cases following a confirmation by an operator. It is also possible that the differences are determined solely automatically following an input of the times by an operator or a transfer of the times from an external storage medium.

The C-arm system can also be embodied for at least partially automatic and/or manual rotation and therefore for recording images in accordance with the differences, stored in a control device, of the determined times t1, t2 and t3. The C-arm can therefore be rotated around the patient automatically or, alternatively, through manually controlled support by an operator in order thereby to record different two-dimensional projections. In this way it is possible to produce three-dimensional angiography images by means of a subsequent reconstruction. The image is recorded in this case in accordance with the differences or delays, stored in the control device, of the times for the injection as well as for the arterial and venous phase and where appropriate further phases. For this purpose an automatic acquisition protocol can be stored in the control device, said protocol automatically carrying out the examination as a function of the determined times.

### BRIEF DESCRIPTION OF THE DRAWINGS

Further advantages, features and details of the invention may be derived from the following exemplary embodiments together with reference to the drawings, in which:

FIG. 1 shows a flow diagram of a method according to the invention, and

FIG. 2 shows a rotational angiography device according to the invention.

### DETAILED DESCRIPTION OF THE INVENTION

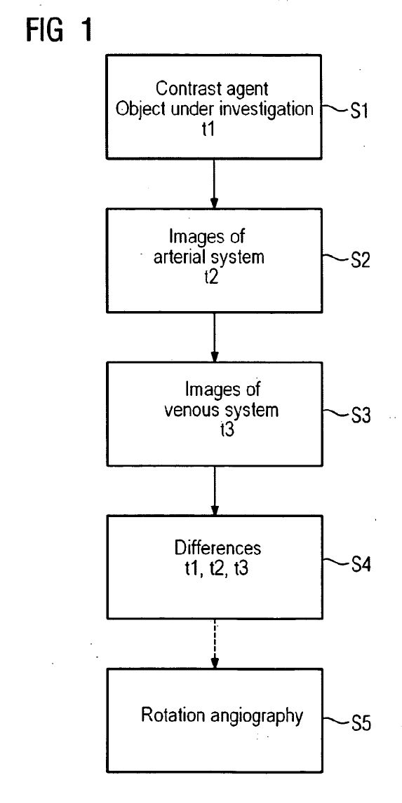

FIG. 1 shows a flow diagram of a method according to the invention. Here, in order to determine the dispersion behavior of a contrast agent bolus in the blood vessel system, contrast agent is first introduced into same. This takes place at time t1 with the aid of a suitable injector.

Next, in step S2, preparatory images are produced as test images which show at least a part of the vascular system of the object under investigation in order to reveal the time curve of the uptake of the contrast agent bolus in the arterial vascular system. From the influence of the contrast agent on the recorded images through discoloration and the like, a time t2 for recording a rotational angiography image of an arterial phase is determined, said time being specified by the uptake in the arterial vascular system. If appropriate, test images of different arterial phases can be produced, for example of an early arterial and a late arterial phase.

Preparatory images are also produced which show the time curve for the dispersion of the contrast agent bolus in the venous vascular system, as depicted in step S3. The images recorded in steps S2 and S3 may possibly belong to a single sequence and the order of observation of the respective phases can vary as a function of the injection and the number of phases to be observed.

In each case at least one further time t3 specifying the time for recording a rotational angiography of a venous phase is determined in step S3, said time t3 in turn being determined on the basis of the changes in the sequence of test images.

Since the individual dispersion characteristics of the contrast agent for the particular patient are dependent on the time of introduction of said contrast agent, the differences of the determined times t1, t2 and t3 as well as possibly further differences for further determined times are determined in the following step S4. The amounts of the differences are stored or noted for the subsequent rotational angiography.

In the following step S5, finally, the planned rotational angiography is optionally performed, wherein the individual delays determined for the patient can be set in a control device of the C-arm system provided for this purpose. In this way optimal image results are obtained in which in particular the separation of the individual vessels into arteries and veins is ensured. In the process, in the course of recording the test images according to steps S2 and S3, image processing and/or evaluation functions such as, for example, subtraction angiography are used in order to determine the times in as error-free a manner as possible.

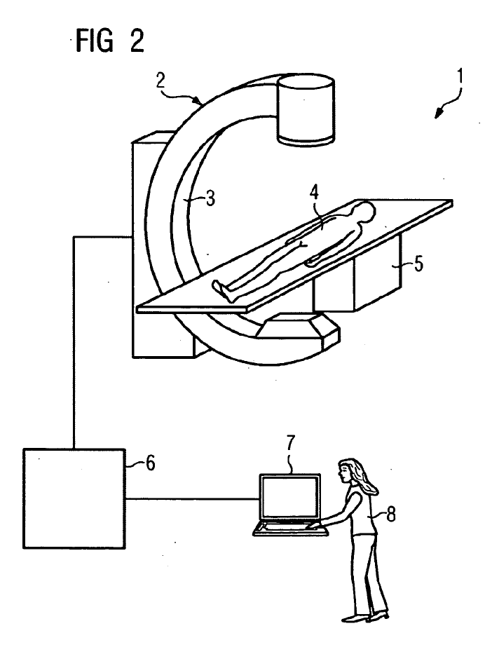

Finally, FIG. 2 shows an inventive rotational angiography device 1 which has an x-ray based C-arm system 2. The C-arm 3 of the C-arm system 2 can be rotated around the patient 4 who is situated on a patient table 5, with in each case two-dimensional projections being recorded, from which the anatomical structure of interest can be reconstructed three-dimensionally by means of suitable algorithms.

The rotational angiography device 1 also has a control device 6 which is connected to an image output means 7 which, in addition to a display, also has an input device for an operator 8.

Preparatory images of the patient 4 which at least partially show the vascular system are generated with the aid of the rotational angiography device 1, which can have further C-arms 3 not shown here. Following the injection of a contrast agent, times which are optimal for recording rotational angiography images of different phases are determined from the preparatory images. Toward that end, the image data is forwarded by the C-arm system 2 to the control device 6 and evaluated there with image processing and evaluation means in order to derive the corresponding times for the rotational angiography images from the change in color during the uptake of the contrast agent bolus in the arterial or venous system. For this purpose functions are used which are dependent on the part of the body to be represented or the region of interest. For example, during the recording of an image of a static part of the body of the patient 4, such as the skull, subtraction angiography can be performed in order to determine the optimal time for recording an arterial or venous phase.

The amounts of the differences are then formed from the times and stored in a memory unit for access by the control device 6. The differences or times can be displayed to the operator 8 on the image output means 7, whereupon the operator 8 can make manual changes if necessary. The operator 8 can simply confirm the times or differences if these appear plausible to him and thereby start the execution of an automatic acquisition protocol after the rotational angiography procedure has been performed with the aid of the C-arm system 2. Thus, with the rotational angiography device 1 according to the invention, the dispersion behavior of the contrast agent bolus is not merely estimated or specified on the basis of empirical values, but optimally determined specifically for the individual patient 4 according to his or her particular condition. By this means high-quality imaging results can be achieved using the rotational angiography device 1 according to the invention.

1-16. (canceled)

17. A method for determining a dispersion behavior of a contrast agent in a blood vessel system of an object in a rotational angiography procedure, comprising:
introducing the contrast agent into the blood vessel system of the object;
determining a time t1 for the introduction;
generating a preparatory image of at least a part of the blood vessel system having an arterial vascular system and a venous vascular system;
defining a time curve for an arterial uptake of the contrast agent in the arterial vascular system according to the preparatory image;
determining a time t2 for recording a rotational angiography image of an arterial phase based on the defined time curve;
defining another time curve for a venous uptake of the contrast agent in the venous vascular system according to the preparatory image;
determining a time t3 for recording a rotational angiography image of a venous phase based on the defined another time curve; and
calculating differences in the times t1, t2 and t3 for performing the rotational angiography procedure.

18. The method as claimed in claim 17, wherein the time curves are determined by tracking a coloration induced by the contrast agent.

19. The method as claimed in claim 17, wherein the preparatory image is generated at least partially using a subtraction angiography.

20. The method as claimed in claim 19, wherein the subtraction angiography subtracts an image of the object which does not contain the contrast agent from an image of the object which contains the contrast agent.

21. The method as claimed in claim 20, wherein the subtraction angiography is a digital subtraction angiography.

22. The method as claimed in claim 21, wherein the time t2 or t3 is determined automatically by locating a maximum contrast density of the blood vessel system using the digital subtraction angiography.

23. The method as claimed in claim 19, wherein the preparatory image is generated without processing the subtraction angiography.

24. The method as claimed in claim 17, wherein the differences in the times t1, t2 and t3 are stored in a control device.

25. The method as claimed in claim 17, wherein the contrast agent is introduced arterially or intravenously.

26. The method as claimed in claim 17, wherein the contrast agent is introduced manually or automatically.

27. The method as claimed in claim 17, wherein a start of the introduction is determined as the time t1.

28. The method as claimed in claim 17, wherein a further time for recording a rotational angiography image of a further phase is determined to calculate differences with the time t1, t2, and t3.

29. The method as claimed in claim 17, wherein the differences in the time t1, time t2 and time t3 are set at least partially manually in a control device for performing the rotational angiography procedure.

30. The method as claimed in claim 17, wherein the differences in the time t1, time t2 and time t3 are set at least partially automatically in a control device for performing the rotational angiography procedure.

31. The method as claimed in claim 17, wherein the time t1, t2 and t3 or the differences in the time t1, t2 and t3 are input via a user interface of a control device.

32. The method as claimed in claim 17, wherein the introduction of the contrast agent is started automatically.

33. A rotational angiography device for determining a dispersion behavior of a contrast agent in a blood vessel system of an object in a rotational angiography procedure, comprising:
a C-arm system that performs the rotational angiography procedure;
an injector that injects the contrast agent into the blood vessel system; and
a control device that determines:
a time of the injection as time t1,
a preparatory image of at least a part of the blood vessel system that comprises an arterial vascular system and a venous vascular system,
a time curve for an arterial uptake of the contrast agent in the arterial vascular system according to the preparatory image and a time t2 based on the time curve for recording a rotational angiography image of an arterial phase,
another time curve for a venous uptake of the contrast agent in the venous vascular system according to the preparatory image and a time t3 based on the another time curve for recording a rotational angiography image of a venous phase, and
differences in the times t1, t2 and t3 for performing the rotational angiography procedure.

34. The rotational angiography device as claimed in claim 33, wherein the control device determines the times t1, t2 and t3 or the differences in the times t1, t2 and t3 at least partially automatically.

35. The rotational angiography device as claimed in claim 33, wherein the C-arm system performs the rotational angiography procedure according to the differences in the times t1, t2 and t3.

36. The rotational angiography device as claimed in claim 33, wherein the times t1, t2, t3 and the differences of the times t1, t2 and t3 are stored in the control device.
